(19) United States
(12) Patent Application Publication
(10) Pub. No.: US 2020/0286596 A1
(43) Pub. Date: Sep. 10, 2020

(54) GENERATING AND MANAGING CLINICAL STUDIES USING A KNOWLEDGE BASE

(71) Applicant: International Business Machines Corporation, Armonk, NY (US)

(72) Inventors: Hyuna Yang, Somerville, MA (US); Vishrawas Gopalakrishnan, Cambridge, MA (US); Gigi Y.C. Yuen-Reed, Tampa, FL (US)

(21) Appl. No.: 16/291,454

(22) Filed: Mar. 4, 2019

(51) Int. Cl. G16H 10/20; G06F 16/28; G06F 16/23

(52) U.S. Cl. CPC G16H 10/20; G06F 16/2365; G06F 16/282

(57) ABSTRACT

A computer system generates and manages clinical studies. A first collection of clinical documents is analyzed to extract clinical study goals and corresponding clinical study design components. A second collection of documents is analyzed to extract value sets indicating a mapping between clinical concepts and data elements of clinical information. The extracted clinical study design information and value sets are stored in a hierarchical structure within a repository that relates the clinical study goals, design components, concepts, and the data elements. A new clinical study is generated for a desired clinical study goal based on pattern recognition applied to the extracted clinical study design information and value sets in the repository using the repository. Embodiments of the present invention further include a method and program product for generating and managing clinical studies in substantially the same manner described above.

### BACKGROUND

### 1. Technical Field

Present invention embodiments relate to generating and managing clinical studies, and more specifically, to creating and managing clinical studies using a knowledge base.

### 2. Discussion of the Related Art

A clinical study refers to a research investigation of a study goal, such as a new treatment, intervention, or test as a manner to prevent, detect, treat or manage a disease or clinical condition. A clinical study is a research study that uses human volunteers to expand medical knowledge. In order to conduct a clinical study, clinical concepts related to the study goal are mapped to secondary data elements, such as clinical claims or the electronic clinical records of patients. For example, a clinical study researching Type 2 diabetes can be described as a study based on the clinical concepts of “adult” and “diabetes,” which may be mapped to data sets relating to adult patients and diabetic patients.

Designing a clinical study may require clinical expertise, data domain knowledge, and understanding of study design variations to meet specific study goals and use case considerations. To design a study, researchers often rely on published studies, protocols, and guidelines, which typically contain free-text descriptions of clinical concepts. Manually designing clinical studies can be expensive and time-consuming, since subject matter experts must put in a great deal of effort to locate descriptions of clinical concepts and to map clinical concepts to secondary data elements.

### SUMMARY

According to one embodiment of the present invention, a computer system generates and manages clinical studies. A first collection of clinical documents is analyzed to extract clinical study design information including clinical study goals and corresponding clinical study design components. A second collection of documents is analyzed to extract value sets indicating a mapping between clinical concepts and data elements of clinical information, wherein each value set comprises information pertaining to a source of the value set, frequency of use of the value set, user feedback for the value set, and validation of the value set. The extracted clinical study design information and value sets are stored in a hierarchical structure within a repository, wherein the hierarchical structure relates the clinical study goals, the clinical study design components, the clinical concepts, and the data elements of the clinical information. A new clinical study is generated for a desired clinical study goal based on pattern recognition applied to the extracted clinical study design information and value sets in the repository, wherein the new clinical study includes clinical study design components and value sets derived from the repository. Embodiments of the present invention further include a method and program product for generating and managing clinical studies in substantially the same manner described above.

### BRIEF DESCRIPTION OF THE DRAWINGS

Generally, like reference numerals in the various figures are utilized to designate like components.

FIG. 1 is a block diagram depicting a computing environment for creating and managing clinical studies using a knowledge base in accordance with an embodiment of the present invention;

FIG. 2 is a flow chart depicting a method of recommending a clinical study in accordance with an embodiment of the present invention;

FIG. 3 is a flow chart depicting a method of generating a new clinical study in accordance with an embodiment of the present invention;

FIG. 4 is an example of a data structure of a knowledge base in accordance with an embodiment of the present invention; and

FIG. 5 is a block diagram depicting a computing device in accordance with an embodiment of the present invention.

### DETAILED DESCRIPTION

Present invention embodiments relate to generating and managing clinical studies, and more specifically, to creating and managing clinical studies using a knowledge base. A knowledge base is a repository of information that links together various elements of clinical studies in a hierarchical structure. A knowledge base may relate study goals to design components, clinical concepts, and value sets. Design components may include any aspects of a study that define how the study is conducted, such as data sources, control and variable groups, sample sizes, and the like. Clinical concepts may include any terminology describing specific ideas or subjects associated with a clinical goal, and value sets may include any mappings between data elements in a dataset and a clinical concept. For example, a study goal of the diabetes prevalence in an adult population may be associated with a study testing adult diabetes patients (a design component), diabetes (a clinical concept), adult (another clinical concept), and a secondary dataset (a value set) containing anonymized electronic health records that have been coded with International Classification of Diseases, Ninth (ICD-9) codes beginning in 250 (e.g., ICD-9 250.XX), which is a class of clinical codes relating to diabetes mellitus (a value set).

In order to conduct a clinical study using one or more secondary datasets, researchers must spend a substantial amount of time designing the study based on prior experience and related studies. Researchers must then manually map clinical concepts to data elements in secondary datasets. Rather than relying on subject matter experts to manually design clinical studies, present invention embodiments create and manage a knowledge base that stores clinical study designs and mappings between clinical concepts and secondary data elements. This knowledge base enables users to search for information on previous studies. Furthermore, present invention embodiments apply machine learning techniques to a knowledge base in order to recommend new study designs to users via pattern recognition. Since the knowledge base is updated in response to the generation of new study designs, the process of generating new studies itself becomes more efficient as the knowledge base grows. Thus, present invention embodiments provide the benefit of reducing the amount of computational resources (e.g., processing resources, memory resources) required to generate a new study, as the knowledge base itself is expanded and improved with the generation of each new recommendation.

It should be noted that references throughout this specification to features, advantages, or similar language herein do not imply that all of the features and advantages that may be realized with the embodiments disclosed herein should be, or are in, any single embodiment of the invention. Rather, language referring to the features and advantages is understood to mean that a specific feature, advantage, or characteristic described in connection with an embodiment is included in at least one embodiment of the present invention. Thus, discussion of the features, advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages will become more fully apparent from the following drawings, description and appended claims, or may be learned by the practice of embodiments of the invention as set forth hereinafter.

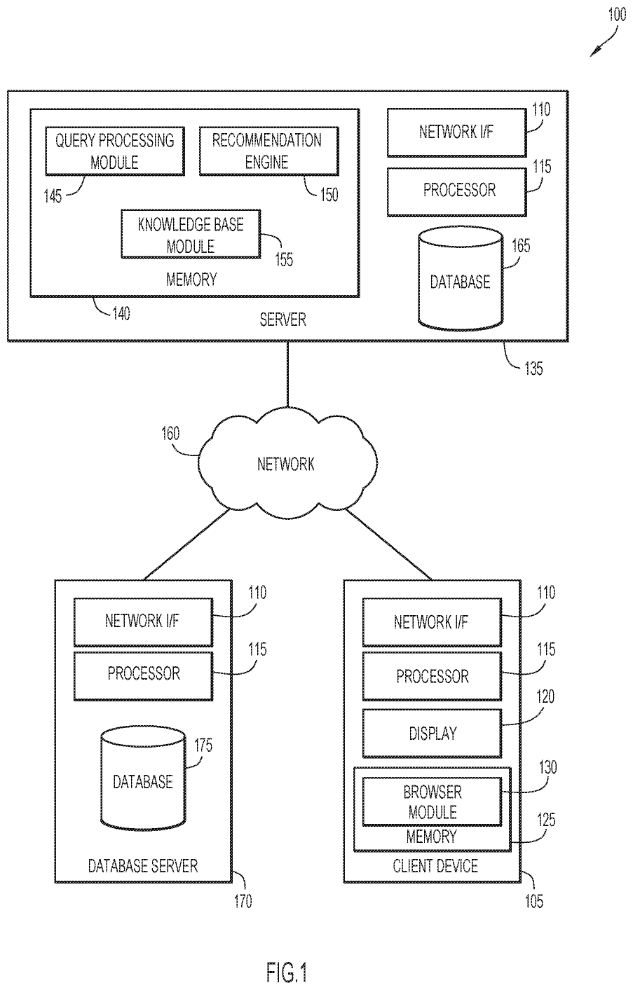

Present invention embodiments will now be described in detail with reference to the Figures. FIG. 1 is a block diagram depicting a computing environment 100 for creating and managing clinical studies using a knowledge base in accordance with an embodiment of the present invention. As depicted, computing environment 100 includes a client device 105, a server 135, and a database server 170. It is to be understood that the functional division among components of computing environment 100 have been chosen for purposes of explaining present invention embodiments and is not to be construed as a limiting example.

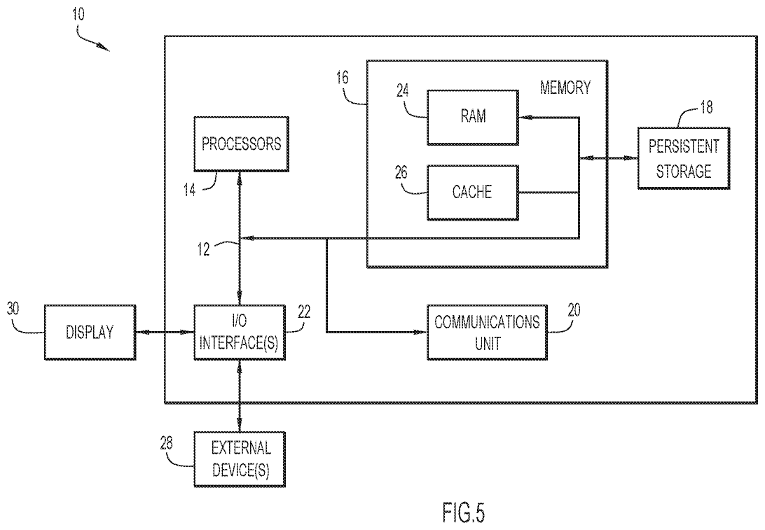

Client device 105 includes a network interface 110, at least one processor 115, a display 120, and memory 125. Memory 125 includes browser module 130. Client device 105 may include a laptop computer, a tablet computer, a netbook computer, a personal computer (PC), a desktop computer, a personal digital assistant (PDA), a smart phone, a thin client, or any programmable electronic device capable of executing computer readable program instructions. Network interface 110 enables components of client device 105 to send and receive data over a network, such as network 160. Client device 105 may include internal and external hardware components, as depicted and described in further detail with respect to FIG. 5.

Display 120 may include any electronic device capable of presenting information in a visual form. For example, display 120 may be a liquid crystal display (LCD), a cathode ray tube (CRT) display, a light-emitting diode (LED) display, an electronic ink display, and the like. Information relating to clinical studies, including generating, managing, and viewing clinical study information, may be presented to a user of client device 105 via display 120.

Browser module 130 may include one or more modules or units to perform various functions of present invention embodiments described below. Browser module 130 may be implemented by any combination of any quantity of software and/or hardware modules or units, and may reside within memory 125 of client device 105 for execution by a processor, such as processor 115.

Browser module 130 may enable a user of client device 105 to view information relating to clinical studies, including information relating to the design and generation of clinical studies. Browser module 130 may forward queries provided by a user to be processed by server 135. For example, a user may input a query to client device 105, such as “ischemia in adults,” which server 135 may then process according to embodiments presented herein. Browser module 130 may receive the results of a query processed by server 135 and present the results to a user of client device 105 via display 120. Browser module 130 may present to a user of client device 105 any data relating to clinical studies, including, but not limited to, study goals, study design components, clinical concepts, and/or value sets.

Network 160 may include a local area network (LAN), a wide area network (WAN) such as the Internet, or a combination of the two, and includes wired, wireless, or fiber optic connections. In general, network 160 can be any combination of connections and protocols known in the art that will support communications between client device 105, server 135, and/or database server 170 via their respective network interfaces 110 in accordance with embodiments of the present invention.

Server 135 includes a network interface 110, at least one processor 115, memory 140, and a database 165. Memory 140 includes a query processing module 145, a recommendation engine 150, and a knowledge base module 155. In various embodiments of the present invention, server 135 may include a laptop computer, a tablet computer, a netbook computer, a personal computer (PC), a desktop computer, a personal digital assistant (PDA), a smart phone, or any programmable electronic device capable of executing computer readable program instructions. Network interface 110 enables components of server 135 to send and receive data over a network, such as network 160. Server 135 may include internal and external hardware components, as depicted and described in further detail with respect to FIG. 5.

Query processing module 145, recommendation engine 150, and knowledge base module 155 may include one or more modules or units to perform various functions of present invention embodiments described below. Query processing module 145, recommendation engine 150, and knowledge base module 155 may be implemented by any combination of any quantity of software and/or hardware modules or units, and may reside within memory 140 of server 135 for execution by a processor, such as processor 115.

Query processing module 145 may process queries relating to clinical studies. A query is provided by a user and typically includes a study goal; for example, a user may provide a query of “hospitalization risk of hypokalemia.” Query processing module 145 may receive queries from browser module 130 of user device 105. Query processing module 145 may process a query by identifying documents and/or document sources that are relevant to the queried study goal. In some embodiments, the query is processed against a knowledge base to locate documents and/or document sources using a string similarity-based ranking mechanism. The documents may be indexed using an inverted index, and query processing module 145 may perform an inverted index look-up to conduct a full-text search of a knowledge base using the query terms. In some embodiments, query processing module 145 performs a fuzzy search to identify study goals in documents that are relevant to the query. A fuzzy search, also known as approximate string matching, is a search that returns results that may not exactly match a queried string, but include variations of the string. A fuzzy search may process a queried term string to return strings that have additional characters, deleted characters, variations in spelling, and the like. For example, if a query includes the term “cot,” a fuzzy search may return results that include the term “cots,” “cost,” “cat,” and the like. When one or more documents and/or document sources pertaining to studies similar to the queried study goal are identified, query processing module 145 may fetch the specifications of the documents for presentation to the user. Thus, the results of a processed query may include one or more specifications of studies whose study goals are similar to a queried study goal.

After sending the results of a processed query to client device 105, query processing module 145 may issue a prompt to determine whether the user finds the returned studies to be satisfactory. If the user indicates that the returned studies are satisfactory, query processing module 145 may retrieve clinical concepts and value sets of the returned studies for presentation to the user, and may also collect feedback from the user. If the user indicates that the returned studies are not satisfactory, an advanced search may be conducted using recommendation engine 150 to generate new clinical study recommendations.

Recommendation engine 150 may recommend new study designs to a user by processing the user's queried study goal against a knowledge base. Recommendation engine 150 may use known or other natural language processing techniques to identify documents pertaining to existing studies that investigate study goals similar to the queried study goal. In some embodiments, recommendation engine 150 performs a similarity analysis by incorporating the semantics of terms in a queried study goal to identify analogous studies. Recommendation engine 150 may index queried terms according to their semantic meaning in order to generalize the terms into categories that can be used to identify analogous studies. For example, if a user queries “hospitalization risk of chronic kidney disease,” then recommendation engine 150 may generalize “hospitalization” to “utilization” and “chronic kidney disease” to “disease,” and may find analogous studies whose study goals can be generalized to “utilization risk of disease” based on pattern recognition. Recommendation engine 150 may perform pattern recognition using known or other machine learning techniques to learn patterns. By analyzing training sets, recommendation engine 150 identifies features that are associated with clinical concepts to establish patterns. For example, recommendation engine 150 may analyze a training set of studies discussing “hospitalization risk” to learn that the studies typically include patients with at least one in-patient (e.g., hospitalization) record. Recommendation engine 150 may then learn via pattern recognition that studies discussing “emergency room visit risk” will typically include an emergency room visitation history. Similarly, recommendation engine 150 may analyze a training set of studies related to a particular disease population to learn that the studies typically include diagnostic codes and/or medication information; thus, to find studies related to chronic kidney disease, recommendation engine 150 may provide recommendations based on a chronic kidney disease diagnostic code and/or chronic kidney disease medications, which can be determined by searching a knowledge base. Recommendation engine 150 may employ various models to perform the learning (e.g., neural networks, mathematical/statistical models, classifiers, etc.). In some embodiments, recommendation engine 150 may identify analogous studies using a distributional semantics approach in which semantic similarity may be determined based on the distributional properties of words in documents, as linguistic items with similar distributions may have similar meanings.

Once recommendation engine 150 has identified analogous studies, recommendation engine 150 may determine the frequencies in which design components corresponding to query terms appear in the identified studies. Recommendation engine 150 may then assign mappings between design components and query terms based on the determined frequency of each term. Recommendation engine 150 may assign mappings using a list of mapped terms. For example, the term “hospitalization” may be mapped to the terms “readmission” and “emergency room,” since both terms imply a hospital stay. If a term is unmapped, then recommendation engine 150 may identify documents in a knowledge base that contain the unmapped term, and may utilize design components in the identified documents in order to recommend a new study design.

Knowledge base module 155 may generate and maintain a knowledge base of information relating to clinical studies. The knowledge base may include documents relating to clinical studies, and value sets that are collected from published literature, publicly available resources, and/or internal resources. In some embodiments, knowledge base module 155 collects value sets from public resources such as a Centers for Medicare and Medicaid Services (CMS) database, an Observational Health Data Sciences and Informatics (OHDSI) database, and the like. Alternatively or additionally, value sets may be compiled by subject matter experts. In addition to electronic health records, each value set may contain information relating to the source of the set (e.g., whether the data is available internally only, has been published, etc.), how often a value set has been used to support clinical studies, and any user feedback relating to a value set. Value sets may be validated by subject matter experts, and the electronic health records contained within value sets may be de-identified in compliance with one or more data privacy statues, such as the Health Insurance Portability and Accountability Act (HIPAA) or the General Data Protection Regulation (GDPR). In some embodiments, value sets may be deleted by a subject matter expert. Knowledge base module 155 may update value sets when a clinical coding system upon which a value set relies is updated. For example, when International Classification of Diseases, Ninth (ICD-9) is updated to International Classification of Diseases, Tenth (ICD-10), knowledge base module 155 may update the knowledge base accordingly to include references the new coding system. For example, value sets that used ICD-9 codes may be updated to include ICD-10 codes in addition to or as a replacement to, the ICD-9 codes. In some embodiments, old and new value sets may be stored in a knowledge with different time stamps.

Knowledge base module 155 may store information in the knowledge base in a hierarchical structure that associates study goals, study design components, clinical concepts, and data elements in the value sets. Thus, knowledge base module 155 may associate all levels of information with each other so that a user may browse the knowledge base to find relevant studies at any desired level. For example, a knowledge base may link the study goal of diabetes prevalence in an adult population to design components from studies testing adult diabetic patients, to the clinical concept of diabetes, and to value sets that include ICD-9 codes 250.XX, which includes ICD-9 codes corresponding to diabetes. Each clinical concept or value set may contain unique information, and may be mapped to multiple studies.

Knowledge base module 155 may assemble a knowledge base by scanning documents, such as publications and/or other references, to extract study goals and study design components. Study goals and design components may be extracted from documents using known or other natural language processing and machine learning techniques. Knowledge base module 155 may perform pre-processing on documents to extract study design components from each clinical study, including literature, clinical trial protocols, guidelines, and common practice. Knowledge base module 155 may remove irrelevant sentences, such as sentences that do not contain any design component terms, and may classify the remaining relevant sentences according to one or more pre-defined study design components. In some embodiments, knowledge base module 155 classifies relevant sentences according to one or more design component categories, including categories based on a data source (for observational studies), a case inclusion or exclusion criterion, a clinical definition for a case, a clinical definition for a control, a control inclusion or exclusion criterion, an end point, a case feature, modifier, and/or exposure, a study duration or index date, a sample size, a statistical analysis method used, a study result, an intervention or treatment received, and a study phase.

Knowledge base module 155 may generalize study goals according to an ontological system, such as the Unified Medical Language System (UMLS). Thus, knowledge base module 155 may similarly generalize two or more different study goals when the goals share the same semantic meaning. In some embodiments, knowledge base module 155 calculates distributional semantics of terms that appear in the processed documents using the UMLS ontology. Knowledge base module 155 may retrieve documents and value sets to be used for the creation of a knowledge base from one or more databases, such as database 175 of database server 170. Knowledge base module 155 may access one or more databases according to a schedule or on an ad hoc basis (such as when a database is updated) in order to update a knowledge base.

Database 165 may include any non-volatile storage media known in the art. For example, database 165 can be implemented with a tape library, optical library, one or more independent hard disk drives, or multiple hard disk drives in a redundant array of independent disks (RAID). Similarly, data on database 165 may conform to any suitable storage architecture known in the art, such as a file, a relational database, an object-oriented database, and/or one or more tables. Database 165 may store data relating to generating and managing clinical studies using a knowledge base, including one or more knowledge bases of information, mapping information, value sets, and the like.

Database server 170 includes a network interface 110, at least one processor 115, and a database 175. In various embodiments of the present invention, database server 170 may include a laptop computer, a tablet computer, a netbook computer, a personal computer (PC), a desktop computer, a personal digital assistant (PDA), a smart phone, or any programmable electronic device capable of executing computer readable program instructions. Network interface 110 enables components of database server 170 to send and receive data over a network, such as network 160. Database server 170 may store documents pertaining to clinical studies in database 175. In some embodiments, database server 170 may be a server for a public resource of clinical information that is accessible by database server 170. Database server 170 may include internal and external hardware components, as depicted and described in further detail with respect to FIG. 5.

Database 175 may include any non-volatile storage media known in the art. For example, database 175 can be implemented with a tape library, optical library, one or more independent hard disk drives, or multiple hard disk drives in a redundant array of independent disks (RAID). Similarly, data on database 175 may conform to any suitable storage architecture known in the art, such as a file, a relational database, an object-oriented database, and/or one or more tables. Database 175 may store data relating to clinical research, including value sets, which may include one or more secondary datasets (e.g., datasets of de-identified electronic health records). Database 175 may also store documents and other data relating to clinical research, such as information published to scientific journals.

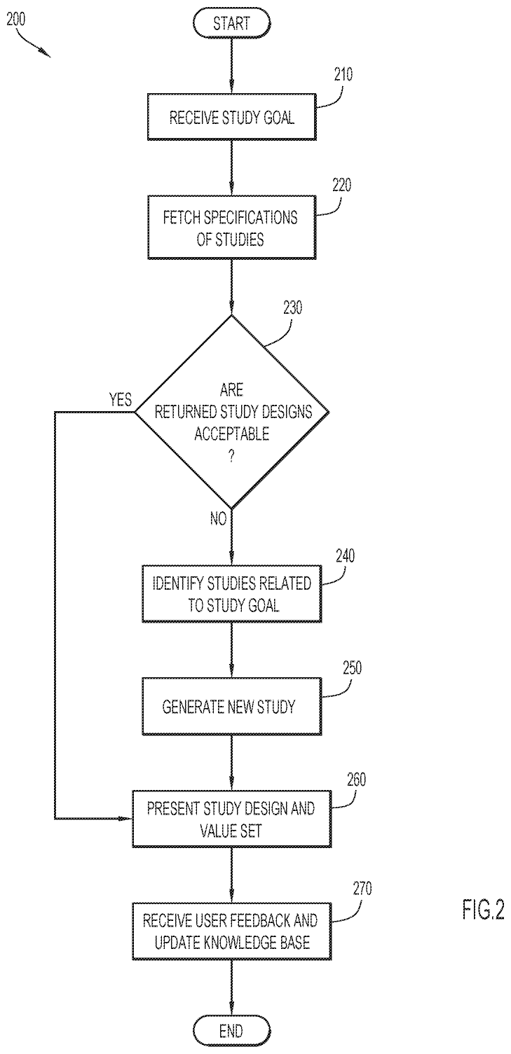

FIG. 2 is a flow chart depicting a method 200 of recommending a clinical study in accordance with an embodiment of the present invention.

A query comprising a study goal is received at operation 210. A query may be input by a user to a computing device, such as client device 105, which transmits the query to server 135 for processing. A query may include any clinical study goal that a user is interested in researching. For example, a study goal may be “hospitalization risk of chronic kidney disease,” “diabetes prevalence in an adult population,” and the like.

The specifications of one or more studies are fetched at operation 220. When a query is received by server 135, query processing module 145 may process the query by identifying one or more studies related to the query and fetching the specifications of the studies. Query processing module 145 may search a knowledge base to identify studies that are relevant to the queried study goal using a string similarity-based ranking mechanism. In some embodiments, query processing module 145 performs an inverted index look-up on study documents indexed by the knowledge base in order to identify studies related to the queried study goal. Once query processing module 145 has identified studies, query processing module 145 may fetch the specifications of the studies from a database, such as database 165, and send the specifications to client device 105.

Operation 230 determines whether the returned study designs are acceptable to a user. When a user is presented with specifications of studies related to the user's query, the user may be prompted to indicate whether the returned study designs are satisfactory. If the user indicates that the returned studies are satisfactory, then method 200 proceeds to present study designs and value sets to the user at operation 260.

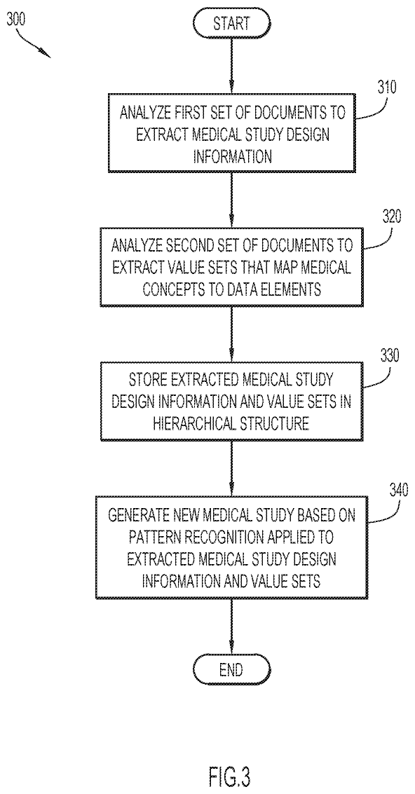

If the user indicates at operation 230 that the returned study designs are not acceptable, then studies that are related to the study goal are identified at operation 240, and a new study is generated at operation 250. Generation of a new study will be depicted and described in further detail below with reference to FIG. 3.

In response to the new study or the user indicating that the returned studies are satisfactory, one or more study designs and value sets are presented to the user at operation 260. Query processing module 145 may extract clinical concepts and value sets from the specifications of the identified studies for presentation to the user. In some embodiments, query processing module 145 identifies clinical concepts using keywords, such as UMLS terminology.

User feedback relating to the study design and value set is received at operation 270, and the knowledge base is updated accordingly. Feedback from a user may include suggestions, such as removing or deleting a suggested result from a result set. In some embodiments, feedback may include the order of a user's selections; for example, a user may select to display a second study design, then a first study design, and then a third study design. The knowledge base may then be updated based on user feedback. In particular, study designs that are disfavored by a user may be down-ranked in future result sets, and subject matter experts may follow up on user feedback to remove study designs from certain result sets.

FIG. 3 is a flow chart depicting a method 300 of generating a new clinical study in accordance with an embodiment of the present invention.

A first set of documents is analyzed to extract clinical study design information at operation 310. The first set of documents may include clinical study documents whose study goals are similar to the queried study goal. Recommendation engine 150 may select documents to be included in the first set of documents by identifying any documents that have similar study goals by analyzing the semantic meanings of terms in the queried study goal to identify analogous studies. In particular, recommendation engine 150 may index queried terms according to their semantic meaning in order to generalize the terms into categories that can be used to identify analogous studies. For example, if a queried study goal is “hospitalization risk of chronic kidney disease,” the goal may be generalized to “utilization risk of disease,” and other studies, which can also be generalized to “utilization risk of disease,” may be identified as similar. Thus, recommendation engine 150 may identify studies such as “emergency room risk of end state renal disease patients,” “hospitalization risk of elderly patients having type 2 diabetes,” “thirty-day readmission rates of cardiovascular patients,” and the like. Once recommendation engine 150 has identified documents with similar study goals, recommendation engine 150 extracts the study design information, including the study goals as well as design components.

A second set of documents is analyzed to extract value sets that map clinical concepts to data elements at operation 320. The second set of documents may include documents indexed by the knowledge base, and may include any documents having design components that were extracted from the first set of documents at operation 310.

Recommendation engine 150 may map query terms to design components using a list of mappings between terms and design components. If a query term does not appear on the list, recommendation engine 150 may calculate the frequency of design components specific to each term in the user's queried study goal, and based on the frequency of design components, assign mappings between query terms and the design components. If a particular design component frequently appears in a document (e.g., beyond a predetermined threshold), that design component may be selected for inclusion in the new clinical study. For example, if a term of the queried study goal is “hospitalization,” recommendation engine 150 may extract design components that appear frequently in a document, such as “patients having at least one in-patient record in 2014” and “admitted to hospital.” Thus, design components that appear frequently in documents are mapped to query terms. In some embodiments, when recommendation engine 150 identifies a new mapping, the mapping is added to the list of mappings.

Extracted clinical study design information and value sets are stored in a hierarchical structure at operation 330. The knowledge base may store study design information (e.g., clinical concepts) and value sets by organizing each element into a hierarchy in which related elements are linked to each other. Design components may be linked to clinical concepts, which are indexed according to an ontology, such as the UMLS ontology. Further, clinical concepts may be linked to value sets containing secondary data that is associated with each clinical concept.

A new clinical study is generated based on pattern recognition that is applied to the extracted clinical study design information and the value sets at operation 340. Recommendation engine 150 may generate a clinical study by using pattern recognition to substitute the design components identified at operation 330 with design components that are relevant for the queried study goal. For example, if a design component includes “readmission rates of diabetic patients” and the queried study goal included “hospitalization risk of chronic kidney disease,” then recommendation engine 150 may use pattern recognition to substitute chronic kidney disease for diabetes, resulting in a recommendation to include “readmission rates of chronic kidney disease” in the new clinical study, along with any value sets relating to kidney disease. Thus, recommendation engine 150 may generate a specification for a new clinical study that is based on a user's queried study goal, including design components for the study, clinical concepts, and value sets.

When a user reviews a new clinical study, a user may provide feedback related to the analogous study designs that have been recommended. The user may explicitly provide feedback by indicating whether a particular design component is useful or not for the user's study goal. Additionally or alternatively, feedback may be collected by tracking the derived specifications and corresponding study goals that are considered by the user. When a user edits a recommended specification, recommendation engine 150 may adjust similarity thresholds that were used in the mapping of query terms to design components.

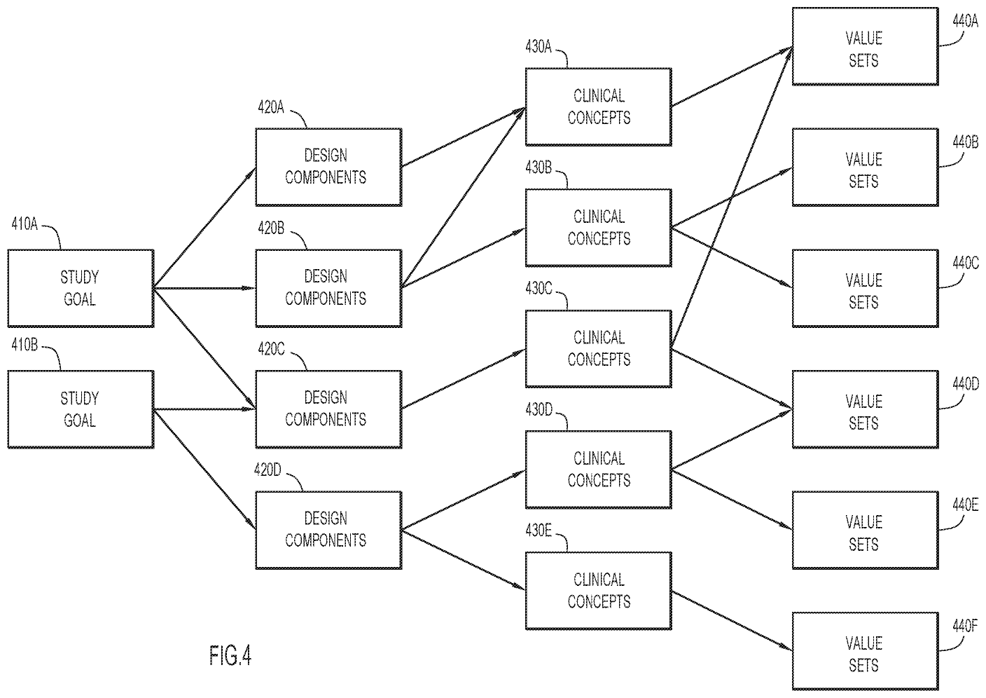

FIG. 4 is an example of a data structure 400 of a knowledge base in accordance with an embodiment of the present invention. As depicted, data structure 400 includes study goals 410A and 410B, design components 420A-420D, clinical concepts 430A-430E, and value sets 440A-440F. Knowledge base module 155 may generate and maintain a knowledge base by adding new study goals, design components, clinical concepts, and/or value sets to the knowledge base, and defining the mappings between the elements. Knowledge base module 155 may update a knowledge base in response to an underlying coding system being updated. For example, when the ICD-9 coding system is updated to the ICD-10 coding system, value sets may be updated accordingly. Knowledge base module 155 may provide each value set with a time stamp, as well as metadata pertaining to the source of the value set, the frequency of use of the value set in studies, user feedback regarding the value set, and any information relating to validation of the value set by a subject matter expert.

FIG. 5 is a block diagram depicting components of a computer 10 suitable for executing the methods disclosed herein. Computer 10 may implement client device 105, server 135, and/or database server 170 in accordance with embodiments of the present invention. It should be appreciated that FIG. 5 provides only an illustration of one embodiment and does not imply any limitations with regard to the environments in which different embodiments may be implemented. Many modifications to the depicted environment may be made.

As depicted, the computer 10 includes communications fabric 12, which provides communications between computer processor(s) 14, memory 16, persistent storage 18, communications unit 20, and input/output (I/O) interface(s) 22. Communications fabric 12 can be implemented with any architecture designed for passing data and/or control information between processors (such as microprocessors, communications and network processors, etc.), system memory, peripheral devices, and any other hardware components within a system. For example, communications fabric 12 can be implemented with one or more buses.

Memory 16 and persistent storage 18 are computer readable storage media. In the depicted embodiment, memory 16 includes random access memory (RAM) 24 and cache memory 26. In general, memory 16 can include any suitable volatile or non-volatile computer readable storage media.

One or more programs may be stored in persistent storage 18 for execution by one or more of the respective computer processors 14 via one or more memories of memory 16. The persistent storage 18 may be a magnetic hard disk drive, a solid state hard drive, a semiconductor storage device, read-only memory (ROM), erasable programmable read-only memory (EPROM), flash memory, or any other computer readable storage media that is capable of storing program instructions or digital information.

The media used by persistent storage 18 may also be removable. For example, a removable hard drive may be used for persistent storage 18. Other examples include optical and magnetic disks, thumb drives, and smart cards that are inserted into a drive for transfer onto another computer readable storage medium that is also part of persistent storage 18.

Communications unit 20, in these examples, provides for communications with other data processing systems or devices. In these examples, communications unit 20 includes one or more network interface cards. Communications unit 20 may provide communications through the use of either or both physical and wireless communications links.

I/O interface(s) 22 allows for input and output of data with other devices that may be connected to computer 10. For example, I/O interface 22 may provide a connection to external devices 28 such as a keyboard, keypad, a touch screen, and/or some other suitable input device. External devices 28 can also include portable computer readable storage media such as, for example, thumb drives, portable optical or magnetic disks, and memory cards.

Software and data used to practice embodiments of the present invention can be stored on such portable computer readable storage media and can be loaded onto persistent storage 18 via I/O interface(s) 22. I/O interface(s) 22 may also connect to a display 30. Display 30 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

Data relating to generating and managing clinical studies (e.g., electronic health records, knowledge base information, mapping information, study goal information, design component information, clinical concept information, value sets, etc.) may be stored within any conventional or other data structures (e.g., files, arrays, lists, stacks, queues, records, etc.) and may be stored in any desired storage unit (e.g., database, data or other repositories, queue, etc.). The data transmitted between client device 105, server 135, and/or database server 170 may include any desired format and arrangement, and may include any quantity of any types of fields of any size to store the data. The definition and data model for any datasets may indicate the overall structure in any desired fashion (e.g., computer-related languages, graphical representation, listing, etc.).

Data relating to generating and managing clinical studies (e.g., electronic health records, knowledge base information, mapping information, study goal information, design component information, clinical concept information, value sets, etc.) may include any information provided to, or generated by, client device 105, server 135, and/or database server 170. Data relating to generating and managing clinical studies may include any desired format and arrangement, and may include any quantity of any types of fields of any size to store any desired data. The data relating to generating and managing clinical studies may include any data collected about entities by any collection mechanism, any combination of collected information, and any information derived from analyzing collected information.

The present invention embodiments may employ any number of any type of user interface (e.g., Graphical User Interface (GUI), command-line, prompt, etc.) for obtaining or providing information (e.g., data relating to generating and managing clinical studies), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

It will be appreciated that the embodiments described above and illustrated in the drawings represent only a few of the many ways of generating and managing clinical studies using a knowledge base.

The environment of the present invention embodiments may include any number of computer or other processing systems (e.g., client or end-user systems, server systems, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system (e.g., desktop, laptop, PDA, mobile devices, etc.), and may include any commercially available operating system and any combination of commercially available and custom software (e.g., server software, networking software, browser module 130, query processing module 145, recommendation engine 150, knowledge base module 155, etc.). These systems may include any types of monitors and input devices (e.g., keyboard, mouse, voice recognition, etc.) to enter and/or view information.

It is to be understood that the software (e.g., server software, networking software, browser module 130, query processing module 145, recommendation engine 150, knowledge base module 155, etc.) of the present invention embodiments may be implemented in any desired computer language and could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The software of the present invention embodiments (e.g., server software, networking software, browser module 130, query processing module 145, recommendation engine 150, knowledge base module 155, etc.) may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information (e.g., data relating to generating and managing clinical studies). The database system may be implemented by any number of any conventional or other databases, data stores or storage structures (e.g., files, databases, data structures, data or other repositories, etc.) to store information (e.g., data relating to generating and managing clinical studies). The database system may be included within or coupled to the server and/or client systems. The database systems and/or storage structures may be remote from or local to the computer or other processing systems, and may store any desired data (e.g., data relating to generating and managing clinical studies).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create mechanisms for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

1. A computer-implemented method of generating and managing clinical studies comprising:
analyzing, via a processor, a first collection of clinical documents to extract clinical study design information including clinical study goals and corresponding clinical study design components;
analyzing, via the processor, a second collection of documents to extract value sets indicating a mapping between clinical concepts and data elements of clinical information, wherein each value set comprises information pertaining to two or more of: a source of the value set, frequency of use of the value set, user feedback for the value set, and validation of the value set;
storing, via the processor, the extracted clinical study design information and value sets in a hierarchical structure within a repository, wherein the hierarchical structure relates the clinical study goals, the clinical study design components, the clinical concepts, and the data elements of the clinical information; and
generating, via the processor, a new clinical study for a desired clinical study goal based on pattern recognition applied to the extracted clinical study design information and value sets in the repository, wherein the new clinical study includes clinical study design components and value sets derived from the repository.

2. The computer-implemented method of claim 1, further comprising:
updating, via the processor, the repository based on user feedback pertaining to the new clinical study.

3. The computer-implemented method of claim 1, further comprising:
generating, via the processor, a second clinical study for a second desired clinical study goal based on a search for clinical studies within the repository corresponding to the second desired clinical study goal, wherein the second clinical study includes clinical study design components and value sets from the clinical studies resulting from the search.

4. The computer-implemented method of claim 1, wherein generating the new clinical study further comprises:
identifying, via the processor, clinical studies within the repository having clinical study goals corresponding to the desired clinical study goal;
determining, via the processor, a frequency of clinical study design components of the identified clinical studies corresponding to terms of the desired clinical study goal;
mapping, via the processor, the clinical study design components of the identified clinical studies to the terms of the desired clinical study goal based on the determined frequencies; and
utilizing, via the processor, the mapped clinical study design components for the new clinical study.

5. The computer-implemented method of claim 4, further comprising:
in response to unmapped terms in the desired clinical study goal, identifying documents containing the unmapped terms and utilizing frequently used clinical study design components within the identified documents for the new clinical study.

6. The computer-implemented method of claim 1, wherein generating the new clinical study further comprises:
recommending the new clinical study to a user.

7. A computer system for generating and managing clinical studies, the computer system comprising:
one or more computer processors;
one or more computer readable storage media;
program instructions stored on the one or more computer readable storage media for execution by at least one of the one or more computer processors, the program instructions comprising instructions to:
analyze a first collection of clinical documents to extract clinical study design information including clinical study goals and corresponding clinical study design components;
analyze a second collection of documents to extract value sets indicating a mapping between clinical concepts and data elements of clinical information, wherein each value set comprises information pertaining to two or more of: a source of the value set, frequency of use of the value set, user feedback for the value set, and validation of the value set;
store the extracted clinical study design information and value sets in a hierarchical structure within a repository, wherein the hierarchical structure relates the clinical study goals, the clinical study design components, the clinical concepts, and the data elements of the clinical information; and
generate a new clinical study for a desired clinical study goal based on pattern recognition applied to the extracted clinical study design information and value sets in the repository, wherein the new clinical study includes clinical study design components and value sets derived from the repository.

8. The computer system of claim 7, further comprising instructions to:
update the repository based on user feedback pertaining to the new clinical study.

9. The computer system of claim 7, further comprising instructions to:
generate a second clinical study for a second desired clinical study goal based on a search for clinical studies within the repository corresponding to the second desired clinical study goal, wherein the second clinical study includes clinical study design components and value sets from the clinical studies resulting from the search.

10. The computer system of claim 7, wherein the instructions to generate the new clinical study further comprise instructions to:
identify clinical studies within the repository having clinical study goals corresponding to the desired clinical study goal;
determine a frequency of clinical study design components of the identified clinical studies corresponding to terms of the desired clinical study goal;
map the clinical study design components of the identified clinical studies to the terms of the desired clinical study goal based on the determined frequencies; and
utilize the mapped clinical study design components for the new clinical study.

11. The computer system of claim 10, further comprising instructions to:
in response to unmapped terms in the desired clinical study goal, identify documents containing the unmapped terms and utilizing frequently used clinical study design components within the identified documents for the new clinical study.

12. The computer system of claim 7, wherein the instructions to generate the new clinical study further comprise instructions to:
recommend the new clinical study to a user.

13. A computer program product for generating and managing clinical studies, the computer program product comprising a computer readable storage medium having program instructions embodied therewith, the program instructions executable by a computer to cause the computer to:
analyze a first collection of clinical documents to extract clinical study design information including clinical study goals and corresponding clinical study design components;
analyze a second collection of documents to extract value sets indicating a mapping between clinical concepts and data elements of clinical information, wherein each value set comprises information pertaining to two or more of: a source of the value set, frequency of use of the value set, user feedback for the value set, and validation of the value set;
store the extracted clinical study design information and value sets in a hierarchical structure within a repository, wherein the hierarchical structure relates the clinical study goals, the clinical study design components, the clinical concepts, and the data elements of the clinical information; and
generate a new clinical study for a desired clinical study goal based on pattern recognition applied to the extracted clinical study design information and value sets in the repository, wherein the new clinical study includes clinical study design components and value sets derived from the repository.

14. The computer program product of claim 13, further comprising instructions to cause the computer to:
update the repository based on user feedback pertaining to the new clinical study.

15. The computer program product of claim 13, further comprising instructions to cause the computer to:
generate a second clinical study for a second desired clinical study goal based on a search for clinical studies within the repository corresponding to the second desired clinical study goal, wherein the second clinical study includes clinical study design components and value sets from the clinical studies resulting from the search.

16. The computer program product of claim 13, wherein the instructions to generate the new clinical study further comprise instructions to cause the computer to:
identify clinical studies within the repository having clinical study goals corresponding to the desired clinical study goal;
determine a frequency of clinical study design components of the identified clinical studies corresponding to terms of the desired clinical study goal;
map the clinical study design components of the identified clinical studies to the terms of the desired clinical study goal based on the determined frequencies; and
utilize the mapped clinical study design components for the new clinical study.

17. The computer program product of claim 16, further comprising instructions to cause the computer to:
in response to unmapped terms in the desired clinical study goal, identify documents containing the unmapped terms and utilizing frequently used clinical study design components within the identified documents for the new clinical study.

18. The computer program product of claim 13, wherein the instructions to generate the new clinical study further comprise instructions to cause the computer to:
recommend the new clinical study to a user.
